(19) United States
(12) Patent Application Publication
(10) Pub. No.: US 2023/0340387 A1
(43) Pub. Date: Oct. 26, 2023

(54) METHODS AND SYSTEMS FOR VACCINE PRODUCTION

(71) Applicant: ADVA Biotechnology Ltd., Kiryat Tivon (IL)

(72) Inventors: Ohad KARNIELI, Kiryat Tivon (IL); Barbara PALDUS, Kiryat Tivon (IL)

(21) Appl. No.: 17/997,711

(22) PCT Filed: Apr. 1, 2021
(86) PCT No.: PCT/IB2021/000208

§ 371 Date: Nov. 1, 2022

Related U.S. Application Data
(60) Provisional application No. 63/003,706, filed on Apr. 1, 2020.

(51) Int. Cl. C12M 1/00; C12M 1/36

(52) U.S. Cl. CPC C12M 23/58; C12M 41/48; C12M 29/10; C12M 29/18

(57) ABSTRACT

A decentralized distributed vaccine manufacturing systems and methods thereof provide a cost effective, simple to operate, automated, and small-scale development and manufacturing process by automated computer-controlled devices. The devices and methods disclosed that allows localized vaccine development and manufacture. The bioreactor systems can include at least one bioreactor chamber, at least one reservoir, a plurality of sensors, and a fluid circuit. The operational methods disclosed herein are directed towards growing cells or tissue while measuring various parameters, and a controlled operation of the various parameters during the operation of the bioreactor systems.

### TECHNICAL FIELD

The exemplary embodiments relate to methods and systems for production of vaccines with a bioreactor system configured for one or more control schemes of a bioreaction.

### BACKGROUND

Coronavirus-19 (COVID-19) has caused millions of deaths worldwide. The World Health Organization (WHO) declared the disease caused by the COVID-19 virus to be pandemic. Beyond the severe repercussions to human health, COVID-19 has caused social and economic lockdowns, which has severely impacted global economic activity. According to the WHO, COVID-19 is not the first widespread disease that the world has faced. It will also not be the last.

The effects of infectious diseases, like COVID-19, vary greatly, ranging from asymptomatic to lethal. Diseases are linked to viruses and bacteria transmitted from animals and insects to humans. Furthermore, in recent years, exotic viruses have become an increasing threat to global health due to rapid growth in global travel. Because of increasing travels, which erode geographic barriers to disease transmission, and concomitantly with the emergence and reemergence of uncommon infectious diseases, front-line clinicians are increasingly more likely to encounter patients with exotic viral infections.

A vaccine is a biological preparation that induces personal immunity against a specific infectious disease. A vaccine typically contains an agent that resembles a disease-causing microorganism and is often made from weakened or killed forms of the microbe/virus, its toxins, or one of its surface proteins. Typically, the agent stimulates the body's immune system to recognize the agent as a threat, destroy it, and promote immune memory toward to potential encounters in the future. Vaccines can be prophylactic (e.g., to prevent or ameliorate the effects of a future infection by a natural or “wild” pathogen), or therapeutic (e.g., vaccines against cancer, which are being investigated). Vaccines are usually developed and manufactured in large scale manufacturing plants with highly trained staff using dedicated facilities and equipment. Thus, the process is usually expensive, and lengthy.

In pandemic cases or biologically contaminated areas, physical segregation is a critical step in stopping the spreading of the disease. Local and de-centralized development and manufacturing of vaccine can be a significant tool to save lives, contain and stop the infection from further spreading.

Generally, bioreactors may be used to culture microorganisms and living cells in a contained and controlled environment. Generally, culturing and processing of such microorganisms and cells may require several steps and the various steps may be performed before and/or during the culturing while monitoring certain parameters.

### SUMMARY

The exemplary embodiments provide a cost effective, simple to operate, automated and small-scale development and manufacturing process by at least one automated computer-controlled device that enables local vaccine development and manufacturing.

In case of a pandemic, with many people affected or obliged to stay at home (e.g., quarantined), there is a risk functional impediment of central vaccine manufacturing system, thus limiting vaccine availability. Furthermore, in remote areas like secluded towns or military units, with limited supply chain options due to war, occasional pandemic, or intentional biological terror, the need for simple and safe vaccine manufacturing options at the point of care, is of clinical importance. The exemplary embodiments address this challenge by providing a small-scale fully controlled and automated cell manufacturing and processing device which can be used to manufacture vaccines in a closed loop, in a cost effective and rapid manner.

In some embodiments, a method comprises a control device located at a first location sending computer-readable instructions for producing a vaccine to a bioreactor system located at a second location; wherein the bioreactor system comprises a bioreactor chamber, at least one reservoir, a plurality of sensors, and a fluid circuit, wherein the fluid circuit comprises a first section of the fluid circuit, wherein the first section fluidly connects the bioreactor chamber to the at least one reservoir, and is configured to flow a first fluid contained in the bioreactor chamber to the at least one reservoir, and a second section of the fluid circuit, wherein the second section fluidly connects the at least one reservoir to the bioreactor chamber, and is configured to flow a second fluid contained in the at least one reservoir to the bioreactor chamber; the bioreactor system receiving the computer-readable instructions, operating the bioreactor system based on the computer-readable instructions, wherein the operating the bioreactor system comprises obtaining sensor measured values for at least three parameters via the plurality of sensors; providing a predetermined setpoint for each of the at least three parameters; comparing the sensor measured values to the predetermined setpoint for the at least three parameters; and controlling the fluid circuit to remove some of the first fluid from the bioreactor chamber, add some of the second fluid to the bioreactor chamber, or a combination thereof, until each of the sensor measured values substantially matches the predetermined setpoint of the at least three parameters.

In some embodiments of the method, the at least three parameters are selected from a level of biological factor concentration contained in the bioreactor chamber; a rate of flow of a first fluid into the at least one reservoir; a rate of flow of a second fluid into the bioreactor chamber; a volume of the first fluid; a pH of the first fluid; a temperature of the first fluid; a level of dissolved oxygen of the first fluid; a level of dissolved CO₂ in the first fluid; a level of HCO₃ in the first fluid; and a level of nutrient in the first fluid.

In some embodiments, the bioreactor system comprises a plurality of biological factors contained in the bioreactor chamber.

In some embodiments, the first fluid is a liquid, a gas, a nutrient, a medium, or a combination there of.

In some embodiments, the second fluid is a liquid, a gas, a nutrient, a medium, or a combination there of.

In some embodiments, the controlling the fluid circuit comprises adjusting one to three of the at least three parameters, wherein the fluid circuit adjusts automatically to the adjusting of the one to three of the at least three parameters.

In some embodiments, the controlling the fluid circuit comprises adjusting all of the at least three parameters, wherein the fluid circuit adjusts automatically to the adjusting all of the at least three parameters.

In some embodiments, the adjusting is simultaneous.

In some embodiments, the bioreactor system is configured to have at least two culturing modes selected from a recirculation culturing mode, a perfusion culturing mode, a batch culturing mode, and a fed batch culturing mode; and the operating the bioreactor system further comprises changing, from one to another, of the at least two culturing modes.

### BRIEF DESCRIPTION OF DRAWINGS

Various embodiments of the present disclosure can be further explained with references to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as necessarily limiting, but merely as representative basis for teaching one skilled in the art to variously employ one or more illustrative embodiments.

FIG. 1 is a schematic block diagram illustrating various components of a bioreactor system in accordance with some embodiments of the bioreactor systems of the present disclosure.

FIGS. 2A, 2B, and 2C are graphs demonstrating results of exemplary embodiments of the bioreactor control system of the current disclosure which are illustrative of some exemplary aspects of at least some embodiments of the present disclosure.

FIGS. 3A, 3B, 3C, and 3D are additional graphs demonstrating results of exemplary embodiments of the bioreactor control system of the current disclosure which are illustrative of some exemplary aspects of at least some embodiments of the present disclosure.

FIG. 4 is a schematic block diagram illustrating various components of a bioreactor system in accordance with some embodiments of the bioreactor systems of the present disclosure.

FIG. 5 is another schematic block diagram illustrating various components of a bioreactor system in accordance with some embodiments of the bioreactor systems of the present disclosure.

FIG. 6 shows graphs demonstrating results of exemplary embodiments of the bioreactor control system of the current disclosure which are illustrative of some exemplary aspects of at least some embodiments of the present disclosure.

FIG. 7 is an exemplary flow chart of an embodiment of the methods disclosed herein.

FIG. 8 is an exemplary schematic diagram illustrating an exemplary deployment of a decentralized system and method in accordance with some embodiments of the bioreactor systems of the present disclosure.

### DESCRIPTION OF EMBODIMENTS

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the term “biological factor” includes, but is not limited to, a cell, a secreted factor, a vaccine, a virus, an exosome, or any combination thereof.

In some embodiments, a vaccine includes proteins, antibodies, mRNA, a partial virus, a full virus, or any combination thereof.

In some embodiments, a vaccine is a protein, antibody, mRNA, a partial virus, a full virus, or any combination thereof.

As used herein, the term “fluid” refers to a liquid or a gas. As used herein, the term “medium” refers to a fluid or combination of fluids in which cells are capable of growing. As used herein, the term “flow” or “flows” refers to fluid that continually deforms under an applied pressure and/or an applied shear stress. Further to this, “a flow rate” as used herein is the rate per amount a time that a substance flow. Also, as used herein, the flow or movement of a substance entering the system (inlet), exiting the system (outlet) and pumps between parts in an exemplary bioreactor system are by any suitable one-way valve that allows the transfer of a substance. As used herein, when two chambers are “fluidly connected,” this means that fluid is capable of flowing back and forth between the two chambers. Further, the term “fluidly connected” can also mean, based on some configurations of some embodiments, that fluid can have directional flow in a particular direction (e.g., from one chamber to another chamber).

As used herein, “feeding of cells” or “cell feeding” refers to introducing material to a bioreactor, where the introduced material facilitates cell growth. As used herein, “waste medium”, “waste product” or “spent waste” is any material secreted by the cells during cell growth that if present in a cell medium, would hinder cell growth.

As used herein, “a batch culturing mode” refers to feeding a bioreactor system with a predetermined amount of medium and the cells using this fresh or new medium. The waste created by this mode of culturing may be expended at the same rate as receiving new medium and a batch mode is completed when all the medium is spent to waste. Recirculation is not done in a batch culture mode.

As used herein, “a fed batch culture mode” is the same as the batch culture mode, however after each cycle where all the fresh medium was spent, new medium is introduced. The cycle continues for a predetermined amount of times. Recirculation is not done in fed batch culture mode.

As used herein, “a perfusion culture mode” refers to equivalent volumes of fluid medium simultaneously fed and removed from a bioreactor system while cells are retained in the bioreactor chamber. This culture mode is constant feeding and constant removal of cell waste product.

As used herein, “a recirculation culture mode” refers to the continuous operation of a bioreactor and the medium circulates between two chambers only.

As used herein, “setpoint” or “setpoints” is a measured state a control system is aiming to reach, and the control system changes parameters to meet a setpoint or a range for a setpoint.

As used herein, “level” or “levels” is a value or a range of values. For example, a “pH level” can mean a particular and specific pH value or a particular pH range.

In the present disclosure, some embodiments relate to a cell culturing processing and manipulation system including bioreactors and bioreactor systems designed for culturing of cells. In some embodiments, an exemplary bioreactor system may be configured to continuously allow all the necessary steps of selecting, culturing, modifying, activating, expanding, washing, concentrating and formulating in one single unit. In some embodiments, the exemplary bioreactor system may regulate various chemical parameters as needed for culturing cells. According to some embodiments, the exemplary bioreactor system may be used in a variety of culturing modes, such as but not limited to, a batch mode, a fed batch mode, a perfusion mode a recirculation mode, or any combination thereof. In some embodiments, the exemplary bioreactor system may be fully controlled in a closed, aseptic environment and may be implemented for a single use (to be disposed after one culturing cycle) as well as for multiple cycle uses.

In some embodiments, any products of the cells in an exemplary bioreactor system may be collected, including, but not limited to, secreted factors (e.g., exosomes, growth factors such as FGF, PDGF, and cytokines such as IL2, TNFalfa), proteins, peptides, antibiotics or amino acids. In some embodiments, an exemplary bioreactor system may provide for optimal and adaptive culturing, wherein manipulation of cells may be performed in a closed system, and wherein the manipulation can be automated. In some embodiments, the high growth density achieved in the exemplary bioreactor system may be 2-fold than that observed using standard culturing conditions. In some embodiments, the high growth density achieved in the exemplary bioreactor system may be 5-fold than that observed using standard culturing conditions. In some embodiments, the high growth density achieved in the exemplary bioreactor system may be greater than 10-fold observed using standard culturing conditions.

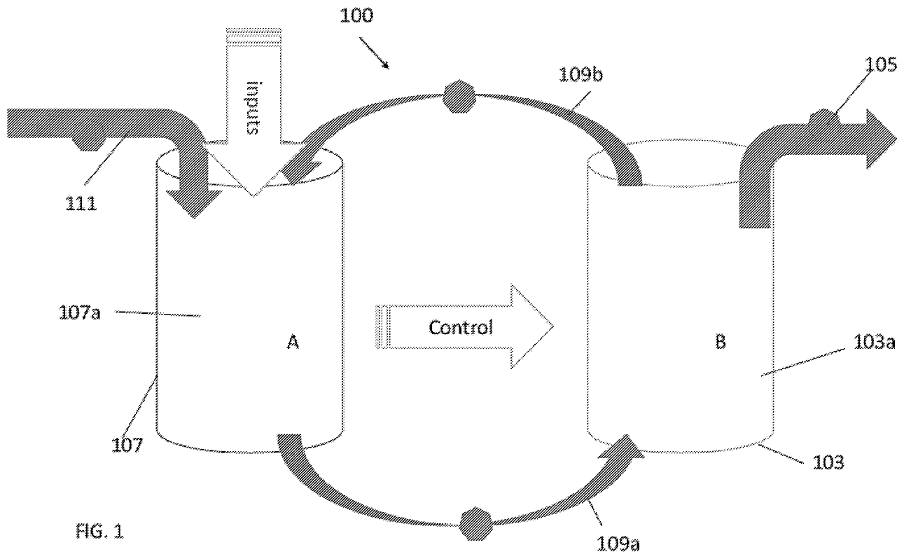

FIG. 1 schematically illustrates an exemplary bioreactor system 100 of the present disclosure. In some embodiments exemplary bioreactor system has a bioreactor 103 which may also comprise an outlet 105. In some embodiments a reservoir 107 is fluidly connected to bioreactor 103 by a fluid circuit (for example, a pump system) comprising at least two sections (e.g., two pumps 109a and 109b). The fluid circuit of the exemplary bioreactor system comprises at least two sections, each, for example, configured with a pump (109a, 109b), and the fluid circuit or the pump system generically will be referred to hereinafter as 109. In some embodiments, the reservoir 107 may further comprise an inlet 111. In some embodiments, the reservoir 107 may be another chamber with fluid medium. In some embodiments, the fluid medium in the reservoir may be the same as the fluid medium in the bioreactor. In some embodiments the fluid medium in the reservoir may be different than the fluid medium in the bioreactor. In some embodiments, the reservoir 107 does not contain any cells. In some embodiments, the reservoir is a container configured to contain a medium for providing delivery of a fluid to the bioreactor 103. In some embodiments, the reservoir is a container configured to receive and contain waste from the bioreactor 103. In some embodiments, the bioreactor system 100 includes at least two reservoirs, a first reservoir 107 configured to contain a medium for providing fluid to the bioreactor 103, wherein the fluid is received via the inlet 111; and a second reservoir (not shown in FIG. 1) configured to receive and contain waste from the bioreactor 103, via a fluid circuit (not shown in FIG. 1) or via the outlet 105.

In some embodiments of the exemplary bioreactor system, the bioreactor 103 further comprises an inner chamber 103a, where the inner chamber 103a is configured to contain at least a plurality of cells. In some embodiments, the bioreactor 103 comprises first fluid medium. In some embodiments, the first fluid medium may comprise at least one gas, at least one nutrient, wherein the at least one nutrient is present in a sufficient amount so as to feed the plurality of cells, a liquid, or any combination thereof. The liquid of the first fluid medium may further comprise a volume of the liquid in the bioreactor 103 In some embodiments, the liquid may have a temperature ranging from 37 Celsius to 42 Celsius. In some embodiments, the liquid may have a temperature ranging from 24 Celsius to 42 Celsius. In some embodiments, the liquid may have a pH level ranging from 6.5 pH to 7.5 pH. In some embodiments, the liquid may have a pH level ranging from 5 pH to 8 pH.

In some embodiments of the exemplary bioreactor system, the reservoir has an inner chamber 107a configured to contain at least a second fluid medium. The second fluid medium may comprise at least one gas, at least one nutrient in a sufficient amount to feed the at least a plurality of cells, a liquid, or any combination thereof. The liquid of the second medium may further comprise a volume of the liquid in the reservoir 107, a temperature and a pH level. Nutrients which may be used for culturing in an exemplary bioreactor system may include, but are not limited to, glucose, lactate, glutamine, glutamate, or a combination thereof. One or more gases that may be used for culturing in an exemplary bioreactor system may include, but are not limited to, oxygen, nitrogen, carbon dioxide, air, or any combination thereof. In some embodiments, the one or more gases are dissolved gases (e.g., dissolved in the medium).

The bioreactor 103, in some embodiments, may further comprise at least two sensors (not shown) configured to measure a plurality of parameters both physical and chemical in a fluid medium and cells contained in the bioreactor. In some embodiments, the bioreactor 103 may further comprise at least three sensors. In some embodiments, the bioreactor 103 may further comprise at least four sensors. In some embodiments, the bioreactor may comprise 5 or more sensors. In some embodiments, the reservoir 107 of the exemplary bioreactor system may contain at least one sensor (not shown) configured to measure parameters both physical and chemical in a fluid medium contained in the reservoir. In some embodiments, the reservoir 107 may further comprise at least 2 sensors. In some embodiments, the reservoir 107 may further comprise at least 3 sensors. In some embodiments, the reservoir 107 may further comprise at least 4 sensors. In some embodiments, the reservoir 107 may further comprise 5 or more sensors.

In some embodiments, the parameters sensed and measured in an exemplary bioreactor system (e.g., via sensors) may be selected from at least, but not limited to: a level of cell concentration; a level of the at least one nutrient; a level of at least one gas; a volume of liquid of the first medium; a pH level of a liquid of the first medium; a temperature of a liquid of the first medium; or any combination thereof.

In some embodiments, the parameters are sensed, detected, measured, controlled, or any combination thereof. In some embodiments of an exemplary bioreactor system, these parameters are selected from at least, but not limited to: a level of cell concentration contained in a bioreactor chamber; a rate of flow of a fluid into a reservoir; a rate of flow of the same or a different fluid into the bioreactor chamber; a volume of at least one fluid; a pH of at least one fluid; a temperature of at least one fluid; a level of dissolved oxygen of at least one fluid; a level of dissolved CO₂ in at least one fluid; a level of HCO₃ in at least one fluid; a level of nutrient in at least one fluid; and any combination thereof.

In some embodiments, the parameters sensed and measured may be, but are not limited to, a temperature, a pH level, a glucose concentration, dissolved oxygen concentration, lactate concentration, glutamine concentration, glutamate concentration, a concentration of dissolved carbon dioxide, a concentration of HCO₃ ions, and any combination thereof.

In some embodiments, at least three of the above parameters are detected by sensors of the bioreactor system. In some embodiments, at least three of the above parameters are measured by the bioreactor system. In some embodiments, at least three of the above parameters are controllable by the configuration of the bioreactor system (e.g., via a control device configured to control a fluid circuit). In some embodiments, an exemplary bioreactor system may control the parameters sensed and measured to a predetermined set point or a predetermined range of that measurement. In some embodiments, the exemplary bioreactor system may control 1 to 5 parameters. In some embodiments, the exemplary bioreactor may control 5 to 10 parameters. In some embodiments, the exemplary bioreactor may control at least three parameters simultaneously, substantially simultaneously, or the input of the control of multiple parameters is not simultaneous but the activation of the fluid circuit in the bioreactor system based on the input of the control does affect changes to these parameters simultaneously.

Returning to FIG. 1, the fluid circuit includes a fluid circuit 109 may be configured such that a first pump 109a extends from the reservoir 107 into the bioreactor 103; and a second pump 109b, extends into the at least one reservoir from the at least one bioreactor. In some embodiments, inlet 111 of reservoir 107 may be used to input materials for bioreactor cultures. In some embodiments outlet 105 of reservoir 107 may be used to remove spent waste, medium, or any combination thereof.

In some embodiments, this configuration of the fluid circuit (e.g., the pump system) 109, in an exemplary bioreactor system, results in the reservoir 107 controlling all of the inputs to the bioreactor via a control device or component thereto. In some embodiments, the reservoir 107 may control the parameters of the bioreaction or cells and a first fluid medium in the bioreactor using the unique pump system in conjunction with a reservoir. In some embodiments, the exemplary bioreactor system measures at least one parameter in the bioreactor and with that measurement may control that parameter by making changes to parameters in the reservoir. In some embodiments of the exemplary bioreactor system, the measurements of parameters are made in the bioreactor 103 and are controlled by making changes to parameters in the reservoir 107. In some embodiments, the exemplary bioreactor system may make changes to parameter or parameters in the reservoir to affect changes in the parameters in the bioreactor 103 by using the pump system 109. In some embodiments of the exemplary bioreactor system, the measurements of parameters are made in the bioreactor 103 and are only controlled by making changes to parameters in the reservoir 107. For example, in some embodiments, the pH level of the bioreactor first medium may be controlled to be at a range of 6.5 pH to 7.5 pH, by controlling the range of the reservoir fluid medium in a range of 5 pH to 8 pH while controlling other set points. In some embodiments, the following parameters, introduced by pump system 109 may also be measured by sensors in either the bioreactor or the reservoir: a rate of flow of liquid of the bioreactor into the reservoir, a rate of flow of liquid from the reservoir into the bioreactor, or any combination thereof.

In some embodiments, an exemplary bioreactor system may require more medium and nutrients and larger culturing volumes. In some embodiments, the exemplary bioreactor system may be configured to allow volume of the bioreactor to be varied and allow additional medium to be added without the need to transfer the cells to a separate container. In some embodiments, the exemplary bioreactor system may adjust volume of medium contained in the bioreactor with the pump system. In some embodiments, the pump system is configured to remove at least some of the liquid from the bioreactor, to add at least some of the reservoir liquid to bioreactor liquid such that the volume of the bioreactor liquid is adjustable, or any combination thereof.

In some embodiments, the exemplary bioreactor system may alternate between culturing modes, as detailed below. In some embodiments, alternating between the culturing modes may facilitate the ability of the bioreactor system to control a plurality of parameters simultaneously.

In some embodiments, the system comprises a batch culturing mode. In some embodiments, the exemplary bioreactor system may process in a batch culturing mode by the reservoir 107 accepting the predetermined amount of medium and pumping 102a the medium to the bioreactor. The bioreactor then may release waste product through outlet 105.

In some embodiments, the system comprises a fed batch culturing mode. In some embodiments, the exemplary bioreactor system may process a fed batch mode by the reservoir 107 accepting the predetermined amount of medium and pumping 102a this medium to the bioreactor. In some embodiments, the process is then repeated for a predetermined amount of time. In some embodiments, the predetermined amount of time is 1 day to two months. In some embodiments the predetermined amount of time is 2 days to 4 months.

In some embodiments, the system comprises a perfusion culture mode. In some embodiments, the exemplary bioreactor system may process a perfusion culture mode by the reservoir 107 accepting through inlet 111 medium and pumping 102a this same medium to the bioreactor. Sensors detect parameters so that only equivalent waste product is removed though outlet 105 of the bioreactor.

In some embodiments, the system comprises a recirculation mode. In some embodiments, the exemplary bioreactor system may process a recirculation culture mode by a first pump 109a continually pumping the liquid medium from the reservoir to the bioreactor and a second pump 109b continually pumping the liquid medium from the bioreactor to the reservoir.

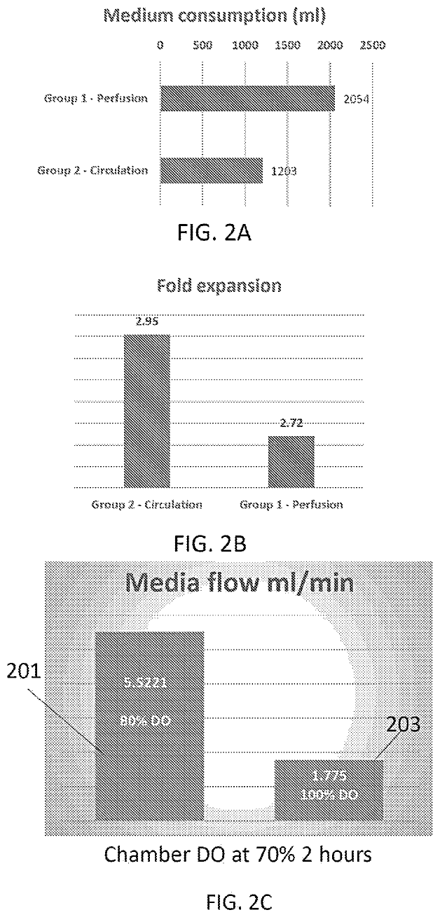

FIGS. 2A, 2B and 2C demonstrate, by way of specific non-limiting examples, cell growth in the same exemplary bioreactor system. For all the specific non-limiting examples of FIGS. 2A, 2B, and 2C, Group 1 is processed in perfusion mode and Group 2 is processed in a recirculation mode. Further, Group 1 is a four-day culture with eighty percent DO (dissolved Oxygen) in the bioreactor 103 and one hundred percent of DO in the reservoir 107. Group 1 seeding was 4.5×10⁷ and harvest was 1.22×10⁸. Group 2 is a four-day culture with eighty percent DO in bioreactor 103 and one hundred percent DO in the reservoir 107. Group 2 seeding was 4.5×10⁷ and the harvest was 1.33×10⁸.

FIG. 2A illustrates specific medium consumption in milliliters of the two groups. FIG. 2B illustrates the fold expansion of the two groups. FIG. 2C illustrates by non-limiting specific example, the controlling of a set point in the bioreactor first medium having more than one option of actions to maintain that set point. Here a setpoint of 70 percent DO for two hours in the bioreactor first medium was achieved by medium (media) from the reservoir being perfused into the bioreactor using pump 109a at a predetermined flow rate of milliliters minute and a predetermined DO percent level in the reservoir. Bar 201 is a flow rate of 5.5221 milliliters per minute with the reservoir at 80 percent DO. Bar 203 is a flow rate of 1.775 milliliters per minute with a 100 percent DO rate. The flexibility of the exemplary bioreactor system is illustrated by this experiment. The multiple options in the reservoir to control at least one same parameter in the bioreactors may be compared and utilized to design improved processes with for example lower sheer forces and efficient media use. In some embodiments, an improved process aimed at yielding high concentration of biological factors (e.g., secreted factors) and/or higher proliferations of cells or the correct phenotype while using efficient medium or resources. In some embodiments, the biological factors (e.g., secreted factors) such as exosomes can be collected via the wastes port once reading the correct density, parameters or time.

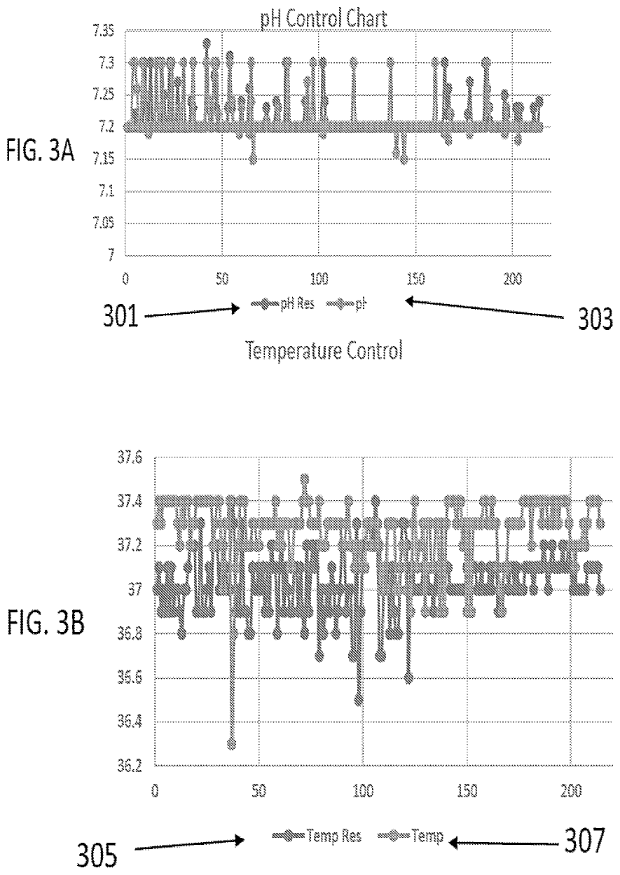

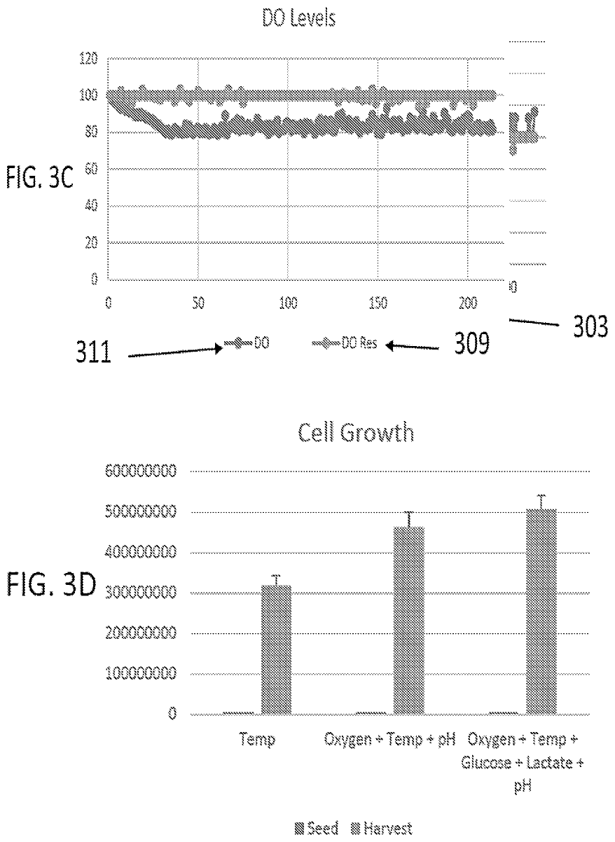

FIGS. 3A, 3B, 3C, and 3D show graphs that demonstrate by specific, non-limiting examples, various parameters that can be controlled in the exemplary bioreactor system. FIG. 3A compares the pH levels and control of the reservoir indicated by 301 with the pH levels in the bioreactor indicated by 303. FIG. 3B compares the temperature and control of the reservoir indicated by 305 with the temperature of the bioreactor indicated by 307. Similarly, FIG. 3C compares the DO levels and control of the reservoir indicated by 309 with the DO levels of the bioreactor indicated by 311. FIG. 3D illustrates by specific non-limiting example the cell growth from seed to harvest when controlling certain parameters that in this specific non-limiting example are: temperature alone; oxygen, temperature and pH level together; and oxygen, temperature, glucose, lactate and pH level together.

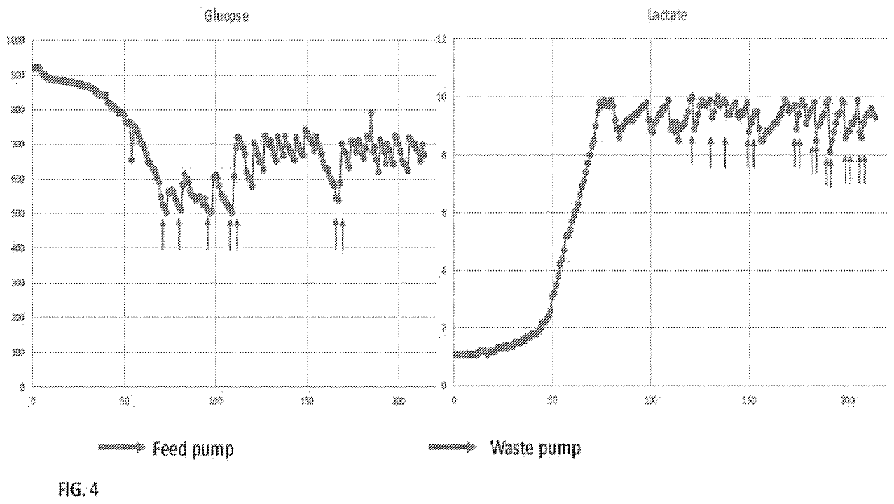

FIG. 4 shows timeline data graphs of yet another embodiment of an exemplary bioreactor system, where the glucose levels and the lactate levels are controlled simultaneously using a method of increasing volume of fluid in the bioreactor. In some embodiments, this method of control can result in more efficient use of medium as illustrated in the graphs of FIG. 4.

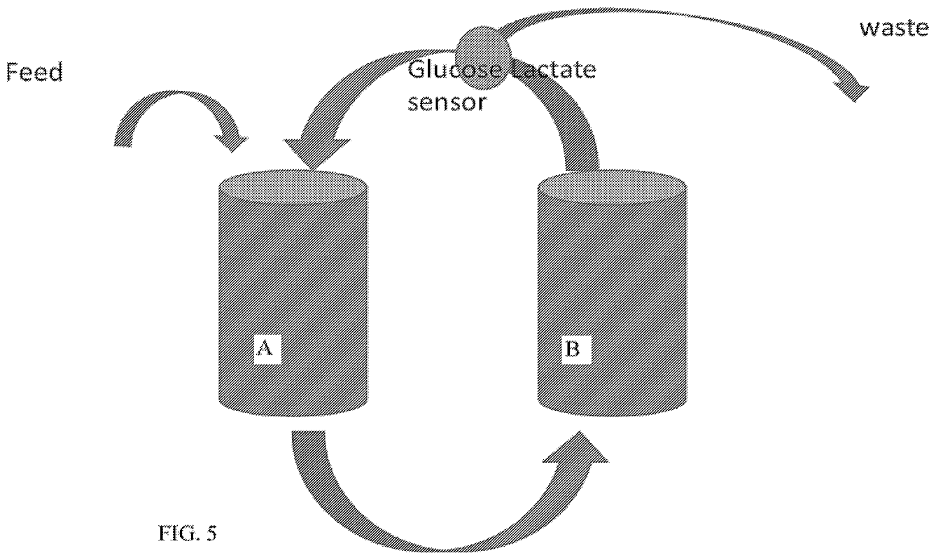

In an alternative embodiment, which is shown in FIG. 5, glucose and/or lactate levels are not measured in chamber A. Instead, glucose and/or lactate levels are only measured on the outlet of chamber B. Consequently, the media in A has a higher glucose level resulting in the media in chamber B at a lower level. In addition, the waste outlet (from B) and the fresh media in (A) is activated based only on the measurement of the media coming out of B.

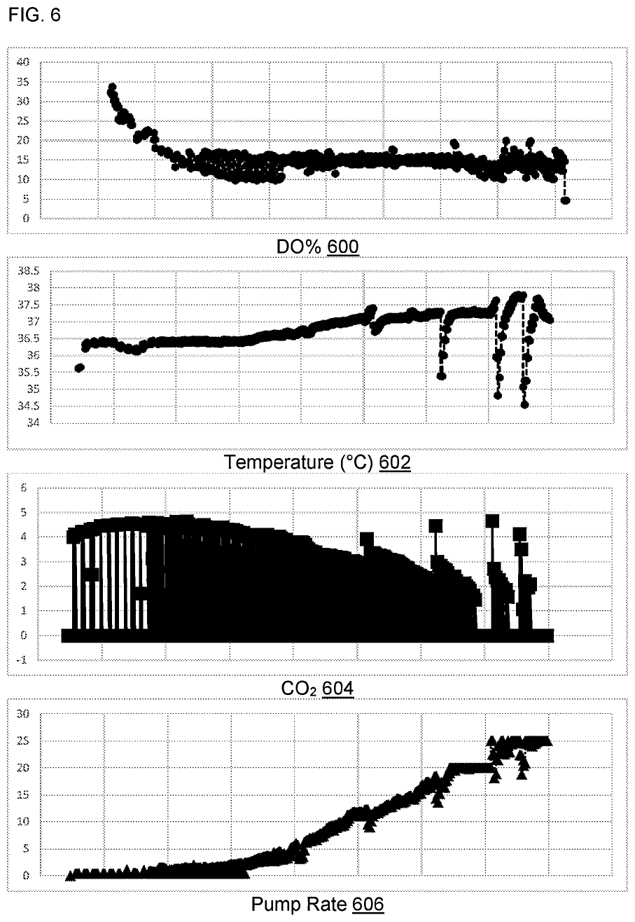

FIG. 6 shows several timeline data graphs 600, 602, 604, 606 according to a particular experimental run using an embodiment of the bioreactor system, having been operated according to the embodiments of the control scheme and methods disclosed herein. In this example, various parameters were detected and measured: dissolved oxygen (DO) of the fluid in the bioreaction chamber; the temperature of the fluid in the bioreaction chamber; the pH of the fluid in the bioreaction chamber; the CO₂ levels of the fluid in the bioreaction chamber; and the flow rate of the fluid flowing into the bioreaction chamber. Over the operational timeline from which these data have been collected, cells contained in the bioreactor system grew. The DO graph (600) shows what happens over time when the setpoint for the DO is set at 15%. The temperature graph (602) shows the changes in the temperature in the bioreaction chamber over time. Also, as shown in graph (604), the CO₂ flow to the reservoir became lower, as there is less need for acid due to cell secretion of lactic acid in the reaction chamber, and there is an increased flow (606) from the reservoir to the reaction chamber over time. Accordingly, as evidenced by the results and data obtained and shown in these graphs, the flow rates, DO level, and CO₂ level in the reservoir can control and affect the DO and pH in the bioreaction chamber. Alternatively, it can be seen that, by controlling the setpoints for the various parameters (e.g., DO), the fluid circuit can be affected automatically to match the measured parameter to these setpoints (e.g., predetermined parameters). That is, it is possible to control, for example, the DO level in the bioreaction chamber by controlling (e.g., changing) the flow rates. Further, it is recognized that, over time, as there are more cells that are contained in the bioreaction chamber, the less CO₂ in needed in the reservoir due to increased flow rates and increased acid secretion.

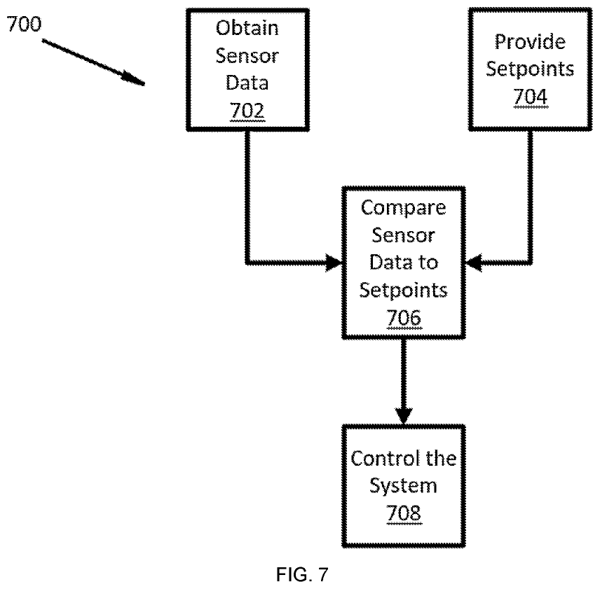

FIG. 7 shows a flow chart of an embodiment of the methods disclosed herein. The embodiment of the method 700 comprises obtaining 702 sensor measured values for at least three parameters via the plurality of sensors. The method 700 also includes providing 704 a predetermined setpoint for each of the at least three parameters. In some embodiments, obtaining 702 and the providing 704 can be in any sequential order. In some embodiments, the obtaining 702 is performed before the providing 704 is performed. In some embodiments, the obtaining 702 is performed after the providing 704 is performed. In some embodiments, the obtaining 702 and the providing 704 are performed at or near the same time (i.e., simultaneously). Then, according to the embodiment shown in FIG. 7, the method 700 comprises comparing 706 the sensor measured values to the predetermined setpoint for the at least three parameters, and then controlling 708 the fluid circuit to: remove some of the first fluid from the bioreactor chamber, add some of the second fluid to the bioreactor chamber, or a combination thereof, until each of the sensor measured values substantially matches the predetermined setpoint of the at least three parameters. In some embodiments, other actions are taken to affect changes to the bioreactor system's operation based on the setpoints and the sensor measured values of the parameters.

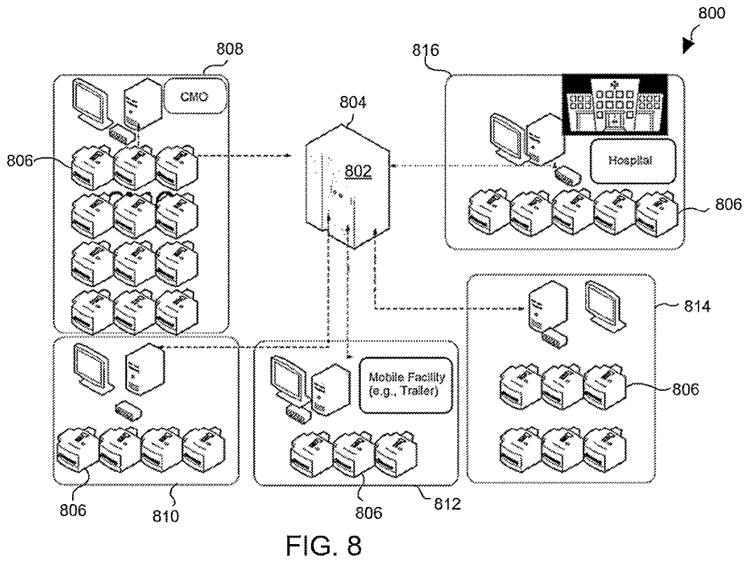

FIG. 8 shows an exemplary decentralized system 800 configured to enable decentralized manufacturing. The decentralized system 800 includes a control unit 802 at a first location 804, which is in communication with at least one bioreactor systems 806 at remote manufacturing location(s) 808, 810, 812, 814, 816. The first location 804 and the remote manufacturing locations 808, 810, 812, 814, 816 are separated and distant from each other. In some embodiments, the remote manufacturing location 808 is a contract manufacturing organization (“CMO”). In some embodiments, the remote manufacturing location 812 is a mobile manufacturing facility, such as, for example, a truck trailer. In some embodiments, the remote manufacturing location is a hospital 816. In some embodiments, all of the bioreactor systems 806 located at the remote manufacturing location(s) 808, 810, 812, 814, 816 are all under centralized control by the control unit 802.

The control unit 802 is configured to communicate computer-readable instructions to the bioreactor systems 806 at the remote manufacturing location 808, 810, 812, 814, 816. The bioreactor systems 806 at the remote manufacturing location 808, 810, 812, 814, 816 are configured to receive the computer-readable instructions sent by the control unit 802.

In some embodiments, by utilizing remote access, vaccine manufacturing of the bioreactor systems 806 can be monitored and controlled by a control unit 802 at a central agency such as the CDC, the FDA, or a company, thereby ensuring the quality and safety of the process.

In some embodiments, the decentralized system 800 can be used in remote areas, and can be placed in hospitals, field hospitals, pharmaceutical facilities, or even trucks, ships or planes close an area where an outbreak is occurring.

In some embodiments, the decentralized system 800 enables military units dealing with biological terror or large pandemics to develop and manufacture vaccines at need and to set up a remote field manufacturing facility that can rapidly and dramatically reduce the time required to develop and manufacture vaccines, decrease costs, and save lives.

In some embodiments, in the case of a pandemic outbreak such as the COVID-19 outbreak an exemplary system 800 can be placed at central hospitals, allowing rapid local vaccine manufacturing from host cells or from patients' B cell hybridoma, making passive immunization quickly available. In some embodiments, in the case of newly identified pathogens such as exotic diseases that can potentially cause a future pandemic outbreak, an exemplary system enables easy and cost-efficient vaccine development and manufacturing upon detection.

In some embodiments, an exemplary single use kit that is part of an exemplary system 800 includes all of the needed manufacturing steps contained within it and all needed raw materials. In some embodiments, the automated nature of the system further simplifies use, and knowledge of how to use the system can be easily acquired. As a result, the exemplary embodiments minimize health risk to the operator.

In some embodiments, an exemplary single-use chamber has two main operating formats. In some embodiments, the first operating format is a suspension-based system using cells such as B cells, T cell, Chinese hamster ovary (“CHO”) cells, and others. In some embodiments, the second operating format is an adherent macro-carrier system allowing the use of adherent cells like primary cells such as mesenchymal stem cells (“MSC's”).

In some embodiments, due to the small scale, safety, and ease of use of use, the exemplary system 800 can serve as an ideal platform for rapid vaccine development and testing even in large pharmaceutical vaccine manufacturing companies.

In some embodiments, due to the small scale, safety, and ease of use of use, the exemplary system can serve as an ideal platform for rapid vaccine development and testing even in large pharmaceutical vaccine manufacturing companies.

The exemplary embodiments enable rapid, simple, contained, cost effective, small scale, decentralized vaccine development and manufacturing system for point of care. Consequently, the exemplary embodiments have the potential to change the way the world can fight biological threats in a rapidly changing global health environment. The exemplary embodiments can serve as a rapid and cost-efficient development and production platform, assisting in the fight against pandemics in remote areas where infections like COVID-19 are identified.

In some embodiments, the exemplary manufacturing station enables small-scale vaccine production at the point of an outbreak. In some embodiments, an exemplary manufacturing station includes a fully contained, end-to-end-small scale cell manufacturing system that is adapted to perform autologous cell therapy manufacturing. In some embodiments, an exemplary system includes a benchtop device with integrated incubator, bioreactors, sensors, cooling chamber, valves and pumps and a customizable control system with a closed and sterile single-use kit. In some embodiments, as a fully contained and automated cell manufacturing system, the exemplary system can be modified to allow manufacturing virus particles or antibodies from host cells at high density, followed by the isolation of the product with an integrated downstream cleaning system such as a system that uses tangential force filtration (“TFF”).

Rather than being a long and costly process using small-scale tools for full development that are limited and manual, resulting in inefficient development process, the exemplary embodiments provide a closed, simple automated end to end device can simplify the vaccine development stage, thereby reducing and expediting the development time line. Furthermore, the cost saving, and effective small-scale development enabled by the exemplary embodiments will allow development and production of vaccines for rare and exotic diseases, enhancing overall healthcare and reducing mortality rate in case of a pandemic outbreak.

While one or more embodiments of the present disclosure have been described, it is understood that these embodiments are illustrative only, and not restrictive, and that many modifications may become apparent to those of ordinary skill in the art, including that various embodiments of the inventive methodologies, the inventive systems, and the inventive devices described herein can be utilized in any combination with each other. Further still, the various steps may be carried out in any desired order (and any desired steps may be added and/or any desired steps may be eliminated).

1. A method, comprising:
sending computer-readable instructions from a control device located at a first location for producing a biological factor to a bioreactor system located at a second location,
wherein the second location is remotely distanced from the first location;
wherein the bioreactor system comprises:
a bioreactor chamber,
at least one reservoir,
a plurality of sensors, and
a fluid circuit,
wherein the fluid circuit comprises:
a first section of the fluid circuit,
wherein the first section fluidly connects the bioreactor chamber to the at least one reservoir, and is configured to flow a first fluid contained in the bioreactor chamber to the at least one reservoir, and
a second section of the fluid circuit,
wherein the second section fluidly connects the at least one reservoir to the bioreactor chamber, and is configured to flow a second fluid contained in the at least one reservoir to the bioreactor chamber;
receiving the computer-readable instructions to the bioreactor system;
operating the bioreactor system based on the computer-readable instructions,
wherein the operating the bioreactor system comprises the steps of:
obtaining sensor measured values for at least three parameters via the plurality of sensors;
providing a predetermined setpoint for each of the at least three parameters;
comparing the sensor measured values to the predetermined setpoint for the at least three parameters; and
controlling the fluid circuit to:
remove some of the first fluid from the bioreactor chamber,
add some of the second fluid to the bioreactor chamber, or
a combination thereof,
until each of the sensor measured values substantially matches the predetermined setpoint of the at least three parameters.

2. The method of claim 1, wherein the at least three parameters are selected from:
a level of biological factor concentration contained in the bioreactor chamber;
a rate of flow of a first fluid into the at least one reservoir;
a rate of flow of a second fluid into the bioreactor chamber;
a volume of the first fluid;
a pH of the first fluid;
a temperature of the first fluid;
a level of dissolved oxygen of the first fluid;
a level of dissolved CO₂ in the first fluid;
a level of HCO₃ in the first fluid; and
a level of nutrient in the first fluid.

3. The method of claim 1, wherein the bioreactor system comprises a plurality of biological factors contained in the bioreactor chamber.

4. The method of claim 1, wherein the first fluid is a liquid, a gas, a nutrient, a medium, or a combination there of.

5. The method of claim 1, wherein the second fluid is a liquid, a gas, a nutrient, a medium, or a combination there of.

6. The method of claim 1, wherein the controlling the fluid circuit comprises:
adjusting one to three of the at least three parameters, wherein the fluid circuit adjusts
automatically to the adjusting of the one to three of the at least three parameters.

7. The method of claim 1, wherein the controlling the fluid circuit comprises:
adjusting all of the at least three parameters, wherein the fluid circuit adjusts
automatically to the adjusting all of the at least three parameters.

8. The method of claim 7, wherein the adjusting is simultaneous.

9. The method of claim 1,
wherein the bioreactor system is configured to have at least two culturing modes selected from:
a recirculation culturing mode,
a perfusion culturing mode,
a batch culturing mode, and
a fed batch culturing mode; and
the operating the bioreactor system further comprises:
changing, from one to another, of the at least two culturing modes.
